(19) United States
(12) Patent Application Publication
(10) Pub. No.: US 2010/0047322 A1
(43) Pub. Date: Feb. 25, 2010

(54) Combination antihypertensive wafer

(76) Inventors: Hans-Rainer Hoffmann, Neuwied (DE); Reto Brändli, San Francisco, CA (US); Frank Theobald, Wehr (DE)

Correspondence Address: D Peter Hochberg;D Peter Hochberg Co, 1940 East 6th Street, 6th Floor, Cleveland, OH, 44114, US

(21) Appl. No.: 12/308,311

(22) PCT Filed: Jun. 4, 2007
(86) PCT No.: PCT/EP2007/004938

§ 371 Date: Dec. 12, 2008

(30) Foreign Application Priority Data
Jun. 16, 2006 (EP) 10 2006 027 794.5

(51) Int. Cl. A61K 9/70; A61K 31/437; A61K 31/522; A61K 31/549; A61K 31/138; A61K 31/404; A61K 31/517; A61K 31/55; A61K 31/551; A61K 31/675; A61P 9/12

(52) U.S. Cl. 424/443; 514/299; 514/263.31; 514/223.2; 514/652; 514/415; 514/252.17; 514/212.07; 514/221; 514/91

(57) ABSTRACT

Sheet-like dosage forms that quickly dissolve or disintegrate in an aqueous environment, for the application of active agent combinations for the treatment of hypertension. The dosage forms contain at least two active agents that are suitable for the treatment of hypertension. The antihypertensive agents are selected from the group that encompasses beta receptor blockers, alpha receptor blockers, calcium antagonists, ACE inhibitors, AT₁ antagonists, centrally acting antihypertensive agents, direct vasodilators, and diuretics. The use of active agent combinations according to the invention for the production of an oral dosage form for the treatment of high blood pressure, a method for the therapeutic treatment of hypertension, and a method for the production of a sheet-like dosage form are also disclosed.

### CROSS-REFERENCE TO RELATED APPLICATIONS

This application is a National Stage application of International Application No. PCT/EP2007/004938, filed on Jun. 4, 2007, which claims priority of German application number 10 2006 027 794.5, filed on Jun. 16, 2006, both of which are incorporated herein by reference in their entireties.

### BACKGROUND OF THE INVENTION

1. Field of the Invention

The present invention relates to sheet-like dosage forms that quickly dissolve or disintegrate in an aqueous environment, and to the application of active agent combinations for the treatment of hypertension.

2. Description of the Prior Art

According to the “Hochdruckliga” (German Hypertension Society), the ideal blood pressure is 120/80 mmHg. By contrast, high blood pressure (hypertension) is considered to be a pathologic elevation of arterial pressure to a systolic pressure value of more than 140 mmHg and a diastolic pressure value of more than 90 mmHg.

Recent studies have found that in countries with a Western lifestyle, hypertension is a wide-spread illness and that in 90% of these sufferers no cause for the elevated blood pressure values can be found, which is why this hypertension is called primary or essential hypertension. Nevertheless, the development of hypertension is promoted by some risk factors such as genetic predisposition, overweight, lack of exercise, stress or high consumption of salt. Primary hypertension, in an above-average number of cases, occurs in association with other illnesses such as overweight, type 2 diabetes, high blood lipid values, and gout, in which cases one refers to metabolic syndrome.

In about 10% of sufferers, however, hypertension is a result of certain basic illnesses, such as narrowing of the renal arteries, chronic renal diseases, as well as changes in hormone balance, or medicaments. This type of hypertension is called secondary hypertension.

In Germany, more than 60 percent of men and 40 percent of women in the age range of 25 to 74 years suffer from hypertension. From age 50, almost every other member of the population suffers from elevated blood pressure.

For the functioning of the body, however, it is of vital importance that the blood pressure constantly remain within a certain range. If blood pressure is too low, the organs may be inadequately supplied with oxygen and nutrients, which limits their functioning and, at the worst, leads to death following complete organ failure.

On the other hand, excessively high blood pressure often leads to arteriosclerosis (arterial calcification) which subsequently leads to apoplexy, cardiac infarction, cardiac insufficiency, renal insufficiency and loss of sight.

If elevated blood pressure is not treated, the risk of these late sequelae increases. However, by normalising the blood pressure these sequelae can be avoided.

Furthermore, suddenly occurring, very strong rises in blood pressure may lead to a so-called hypertensive crisis, which is associated with severe dyspnoea and angina pectoris and is to be regarded as an emergency needing urgent treatment.

Though high blood pressure in its initial stage generally shows no or hardly any symptoms and those affected feel comfortable, treatment is urgently necessary because of the possibility of serious late sequelae.

Blood pressure is closely linked to the total amount of blood circulating in the blood vessels, which, in turn, is in direct correlation with the body's water balance, which is regulated by the kidneys. Furthermore, the diameter of the blood vessels has an influence on the blood pressure, so that these factors are to be regarded as starting points for a therapy.

In cases of only slightly elevated blood pressure, initially, simple measures such as taking regular exercise, weight reduction and a low-sodium diet can lower the blood pressure.

If these measures do not lead to the desired success, control and treatment of high blood pressure by means of medication is necessary which requires a long-term and consistent intake of medicaments.

However, the above-mentioned studies have also shown that only about 5% of patients suffering from high blood pressure are receiving optimal drug treatment.

Furthermore, studies have shown that optimal control of high blood pressure in an advanced stage can only be achieved with a combination of two or more active agents, often from different active agent groups.

In the therapy of hypertension, preference is to be given to a pharmaceutical form which on the one hand improves patient compliance and on the other hand enables easy intake during one's normal rhythm of life. For many patients, a disadvantage of the normal application of tablets is that, often, water or the like is needed for swallowing the tablets. This limits the patient's freedom of movement and is considered to be an inconvenience.

Moreover, in hypertensive emergencies it is necessary to provide a quick and effective therapy. The dosage form should therefore be suitable for achieving a rapid release of the active agents and for ensuring a quick onset of action. For this reason, the disintegration of the dosage form and the release of active agents should take place already at the site of application, in the case of orally administrable dosage forms, for example, already in the oral cavity.

In addition, it should be possible to apply the dosage forms in a simple and direct manner in order to facilitate intake even for patients with severe dyspnoea and oppression, or to enable application by a third person, for example to unconscious patients.

### SUMMARY OF THE PRESENT INVENTION

It was therefore the object of the present invention to provide a pharmaceutical form that enables application of active agent combinations for the treatment of hypertension in such a manner that it allows for discrete, easy intake without the use of an additional auxiliary means of application.

Common dosage forms as used for administration of active agents in the treatment of hypertension are tablets, capsules or drops.

Tablets and capsules can be taken easily, but their onset of action is, as a rule, delayed, and the active agents, when absorbed via the gastrointestinal tract, are subject to the “first-pass effect”, so that high initial concentrations of active agent in the tablet or capsule are required.

Moreover, as a rule some liquid is needed to swallow the dosage form, which is not always immediately available. Furthermore, in the case of a hypertensive crisis swallowing may be difficult or impossible, so that application often turns out to be a problem.

It has been found that this object is achieved by sheet-like dosage forms of a hydrophilic polymer film that disintegrates in the oral cavity, wherein the wafers contain at least two active agents that are suitable for the treatment of hypertension.

### DETAILED DESCRIPTION OF THE PRESENT INVENTION

The free bases or the therapeutically active salts of the individual active agents are also suitable as active agents.

The combination of the active agents in the dosage form according to the invention makes it easier for the patient to take both of the active agents. The absorption of the active agents via the oral mucosa, as compared to other peroral dosage forms, affords, for example, the advantages that patients also having difficulty swallowing or patients refusing to take tablets can be administered medicaments via the oral route. In addition, the risk of medication errors is reduced since the patient has to take only one medicament for both of the active agents. Finally, the dosage forms according to the present invention can be packaged in an inconspicuous package so that intake is possible, even in public, in a manner analogous to taking a chewing gum or to taking “films”, which have come into vogue recently. Compliance and therapeutic success are thereby improved.

In particular, the combination of active agents in one dosage form for the treatment of hypertension—wherein antihypertensives of different active agent classes may be combined—affords special advantages. Thus, for example, a quick-acting, potent pharmaceutical product for treating hypertensive emergencies can facilitate administration of the various agents of emergency treatment.

Furthermore, one active agent combination may contain active agents with different mechanisms of action having a synergistic effect, so that as a result of the different physiological activity, it is possible to dose lower amounts of the active agents for treatment of hypertension than would be the case with single-component compositions.

The administration of these active agent combinations in sheet-like dosage forms (wafers) not only enables easy intake but also an exact coordination of the active agent components to each another, so that false dosages because intake has been forgotten or because of double intake of only one active agent, and thereby insufficient therapy of hypertension, do not occur.

With some active agents, a further advantage of transmucosal administration of active agents consists in the circumvention of the gastrointestinal route and hence the avoidance of the “first-pass” effect after peroral administration, i.e. avoidance of the metabolism of a considerable portion of the active agent during the first liver passage, so that the active agent is utilised to a high degree.

With these active agents, loss of active agent due to the first-pass effect does not occur, so that the dosage of the active agents can be lowered correspondingly, which likewise leads to the patient being disburdened, and to improved well-being as a consequence of lower UDE's.

Because of the simple and low-cost manufacture of the wafers it is possible to provide a large number of pharmaceutical products different active agent concentrations.

If the wafer is made up of a laminate, it is possible, for example, to alter only the layer thickness of an active agent-containing layer, or to alter the concentration of the active agent.

On the other hand, pharmaceutical products can be produced which have different active agent contents but the same active agent ratio, simply by means of cutting the surface of the dosage form to different sizes.

Furthermore, because of their flat shape the wafers of the invention, containing the active agent combinations, can be carried along easily, for example in a wallet, and even when one is travelling they are available at once, are easy to take and take effect quickly, both in the therapy of hypertension and in the case of a suddenly occurring hypertensive emergency.

Water-soluble or swellable polymers that are suitable as a base polymer for the hydrophilic water-soluble and/or swellable polymer film are polymers from the group comprising dextran, polysaccharides, inclusive of starch and starch derivatives, cellulose derivatives, such as carboxymethyl cellulose, ethyl cellulose or propyl cellulose, hydroxypropylmethyl cellulose, hydroxypropyl cellulose, sodium carboxymethyl cellulose (e.g. WALOCEL®), methyl cellulose, hydroxyethyl cellulose and hydroxypropylethyl cellulose, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of these polymers. As an alternative, the polymer film may be made of a polyvinyl alcohol-polyethylene glycol graft copolymer.

The proportion of polymer contained in a dosage form according to the invention is preferably 5 to 95%-wt., more preferably 15 to 75%-wt., relative to the dry mass of the dosage form.

The pharmaceutical preparations according to the invention contain at least two active agents that are used for the treatment of hypertension, wherein it is also possible that at least one active agent from each active agent group be contained therein.

The active agents are selected from the group of antihypertensives comprising the beta receptor blockers, the alpha receptor blockers, the calcium antagonists, the ACE inhibitors, the AT1 antagonists, the centrally acting antihypertensive agents, the direct vasodilators, and the diuretics.

In a preferred embodiment the pharmaceutical preparation contains at least two to five, preferably two to three, active agents.

In the case of an active agent combination of two active agents, one of the active agents is preferably selected from the group of the diuretics, and the second active agent is selected from the group of the beta receptor blockers, the ACE inhibitors, the calcium antagonists or the AT₁ receptor antagonists.

In one of the preferred embodiments of the active agent preparation containing three active agents of the group of the antihypertensives, the first active agent preferably is a diuretic, and the second and third active agent of the combination are a beta receptor blocker and a vasodilator, said vasodilator being selected from the group which comprises the calcium antagonists, ACE inhibitors, alpha₁ receptor blockers and the direct vasodilators, or the second and third active agent are an ACE inhibitor and a calcium antagonist, or the second and third active agent are an antisympathotonic and a vasodilator.

In a further preferred embodiment, the pharmaceutical preparations contain at least one active agent that does not belong to the group of the antihypertensives, for instance a sedative. Alternatively, the pharmaceutical preparations may likewise contain a potassium salt to compensate for the loss of potassium caused by the diuretics.

The diuretics which are used in the pharmaceutical preparations of the present invention are selected from the group which comprises xanthine derivatives, osmotic diuretics, carbonic anhydrase inhibitors, thiazides, loop diuretics, potassium-sparing diuretics, aldosterone antagonists or cycloamidine derivatives. The active agents of these diuretics are selected from the group comprising caffeine, theophylline, theobromine, mannite, sorbite, acetazolamide, hydrochlorothiazide, trichlormethiazide, butizide, bendroflumethiazide, bemetizide, mefruside, chlortalidone, xipamide, clopamide, indapamide, furosemide, azosemide, bumetanide, piretanide, torasemide, etozolin, etacrynic acid, methyl clothiazide, metolazone, polythiazide, spironolactone, potassium canrenoate, triamterene and amiloride, as well as pharmacologically acceptable salts and combinations of these active agents

The active agent content of the diuretic in the dosage form is between 0.1 mg to 50 mg, preferably between 0.5 mg to 20 mg and more preferably between 2 mg to 10 mg, per single dose.

The active agents of the beta receptor blockers used in accordance with the invention are selected from the group which comprises alprenolol, oxprenolol, penbutolol, bupranolol, metipranolol, propanolol, nadolol, pindolol, mepindolol, carteolol, carazolol, timolol, sotalol, metoprolol, betaxolol, bisoprolol, atenolol, acebutolol, celiprolol and bopindolol, as well as pharmacologically acceptable salts and combinations of these active agents.

Preferably, bopindolol, bisoprolol and pindolol are used as beta receptor blockers.

The alpha receptor blockers used in accordance with the invention contain active agents which are selected from the group comprising bunazosin, doxazosin, terazosin and urapidil, as well as pharmacologically acceptable salts and combinations of these active agents.

The active agents of the ACE inhibitors are selected from the group comprising benazepril, captopril, cilazapril, enalapril, fosinopril, imidapril, lisinopril, moexipril, perindopril, quinapril, ramipril, spirapril and trandolapril, as well as pharmacologically acceptable salts and combinations of these active agents.

Preferably, cilazapril, enalapril, benazepril, perindopril, spirapril and trandolapril are used as the ACE inhibitors.

The calcium antagonists used in the pharmaceutical preparations of the present invention are selected from the group which comprises calcium antagonists of the verapamil type, of the diltiazem type and the dihydropyridines.

The active agents of the calcium antagonists are selected from the group comprising diltiazem, gallopamil, verapamil, amlodipin, felodipine, isradipin, lacidipine, lercanidipin, nicardipine, nifedipine, nilvadipine, nisoldipine and nitrendipine, as well as pharmacologically acceptable salts and combinations of these active agents.

Preferably, felodipine, lacidipine, lercanidipin, amlodipin and nicardipine are used as calcium antagonists.

The active agents of the AT₁ antagonists are selected from the group comprising candesartan, eprosartan, irbesartan, losartan, olmesartan, telmisartan and valsartan, as well as pharmacologically acceptable salts and combinations of these active agents.

The antisympathotonics used in the pharmaceutical preparations of the present invention are selected from the group which comprises clonidine and methyldopa, as well as pharmacologically acceptable salts and combinations of these active agents.

Active agents used as direct vasodilators are those selected from the group comprising minoxidil and dihydralazine as well as pharmacologically acceptable salts and combinations of these active agents.

To improve the physicochemical properties, for example reduce the brittleness or embrittlement, humectants, such as glycerine, propylene glycol, sorbitol, mannitol, polyethylene glycol, polyglycerol ester and the like, may be added to the film.

In a further embodiment, antioxidants, for example vitamin C (ascorbic acid), ascorbyl palmitate, vitamin E (tocopherol acetate), hydroxybenzoic acid derivatives, may be added to the wafer, in order to stabilise the film and the active agents. Furthermore, acidic and basic ion exchangers may be used as stabilisers.

In further embodiments, further ingredients such as dyes, pigments, taste flavourings, natural and/or synthetic flavouring substances, sweeteners, buffering systems, may be added to the film. In particular, the taste flavourings and flavouring substances can cover the often bad inherent taste or smell of the active agents and/or give the dosage form a pleasant taste, so that the patient's readiness to take the medication is considerably improved.

The addition of buffering systems on the one hand serves to stabilise the film and the active agents against outside influences and during storage; on the other hand, the pH of the dosage form can thereby be adjusted to a physiologically acceptable pH value, so that irritation of mucous membranes is avoided. By using a buffering system, it is also possible to improve the solubility of acidic or basic active agents in the matrix.

The dosage forms according to the invention are configured so as to be thin, for example in the form of a wafer. The thickness of the dosage form is preferably 0.1 to 5 mm, more preferably 0.5 to 1 mm. The lower limit for the thickness of the dosage forms is about 50 μm. The surface area of the dosage form is between 0.09 cm² and 12 cm², preferably between 1 cm² and 8 cm², and more preferably between 3 cm² and 6 cm².

In a further embodiment, the wafers of the present invention contain a disintegrant or a wicking agent, for example a bicarbonate-acid mixture or an aerosil, being activated by contact with a liquid and accelerating the disintegration of the wafer after application thereof, and thereby also accelerating the release of active agent.

In a preferred embodiment, the wafer is present as a foam so that the release of active agent takes place even more rapidly because of the enlarged surface. In this embodiment, the cavities of the foam may contain one or more of the active agents in liquid form.

To improve the absorption of the active agents via the mucous membrane, permeation enhancers, such as substances from the groups of the fatty alcohols, fatty acids, poly-oxyethylene fatty alcohol ethers, polyoxyethylene fatty acid esters, fatty alcohol esters and fatty acid esters, particularly sorbitan monolaurate or esters of long-chain fatty acids with methyl, ethyl or isopropyl alcohol, or esters of fatty alcohols with acetic acid or lactic acid, or substances such as DMSO (dimethyl sulfoxide) and oleic acid diethanolamine may likewise be incorporated in the film. The constituent amount of these substances is 0.1 to 25%-wt., preferably 1 to 10%-wt., in each case relative to the total weight of the active agent matrix.

Furthermore, the composition of the wafer may contain compounds that retard the release of active agent (e.g., mi-croencapsulation), said wafer, in a preferred embodiment, containing a liquid active agent in microencapsulated form. This liquid active agent can, for instance, be an alcoholic nitroglycerin solution.

In a further embodiment, the wafer has mucoadhesive proper-ties, so that it adheres to the mucous membrane until it is completely dissolved.

In a preferred embodiment, at least one of the active agents is bound to an ion exchanger, so that the hydrophilic polymer disintegrates quickly in the oral cavity, whereas the release of active agent is retarded or occurs when the pH has changed, e.g. in the gastrointestinal tract. In this way, active agents having a different mechanism of action and absorption can be administered in one dosage form, that is, at least one of the released active agents is either absorbed at the site of application, for example via the mucous membrane, or it is transported fur-ther and absorbed at another site.

The wafer may also be made up as a laminate with different layers, with the active agents being contained in discrete layers which are spatially separated from each other and differ from each other in terms of their composition. In this way, the active agents can be released at different sites of action, but also with retardation, if the disintegration times of the various layers of the wafer differ from each other.

Likewise, the active agents may be arranged within layers that disintegrate at different rates, so that the preparation as a whole shows a retardation effect.

In a further embodiment, one of the outer layers may be mucoadhesive, to promote the adherence of the dosage form on the mucous membrane and to facilitate the active agent absorption via the mucous membrane by establishing direct contact.

The disintegration of the inventive dosage form in an aqueous medium preferably takes place in the range from 1 s to 5 min, more preferably in a range from 5 s to 1 min, and most preferably in the range from 10 s to 30 s.

The dosage forms according to the invention are advantageously suitable for administering medicaments in the oral cavity or for rectal, vaginal or intranasal administration. They can be used in human medicine as well as in veterinary medicine.

The present invention furthermore relates to the use of an active agent combination according to the invention for the production of an oral dosage form for the treatment of hypertension, said dosage form preferably being formulated as a wafer.

Furthermore, the present invention relates to a method for the therapeutic treatment of a person suffering from high blood pressure, wherein the administration of an above-described active agent combination of antihypertensive agents is carried out by means of an orally applicable dosage form with transmucosal absorption.

Finally, the present invention also relates to a method for the production of a sheet-like dosage form, comprising the following steps:

preparing a solution containing at least one polymer and at least two antihypertensive active agents;

spread-coating the solution on a coating substrate; and

solidifying the spread-coated solution by drying and withdrawing the solvent.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A sheet-like pharmaceutical preparation usable in dosage form which is based on hydrophilic polymers and which quickly disintegrates upon contact with moisture, for the treatment of high blood pressure, the pharmaceutical preparation in dosage form comprising an active agent combination of at least two active agents which are suitable for the treatment of hypertension.

2. The pharmaceutical preparation according to claim 1, wherein said at least two active agents are selected from the group of antihypertensive agents consisting of beta receptor blockers, alpha receptor blockers, calcium antagonists, ACE inhibitors, AT₁ antagonists, centrally acting antihypertensive agents, direct vasodilators and diuretics

3. The pharmaceutical preparation according to claim 2, wherein one of the active agents is selected from the group consisting of the beta receptor blockers, and the second active agent is selected from the group consisting of the diuretics.

4. The pharmaceutical preparation according to claim 2, wherein one of the active agents is selected from the group consisting of the ACE inhibitors, and the second active agent is selected from the group consisting of the diuretics.

5. The pharmaceutical preparation according to claim 2, wherein one of the active agents is selected from the group consisting of the calcium antagonists, and the second active agent is selected from the group consisting of the diuretics.

6. The pharmaceutical preparation according to claim 2, wherein one of the active agents is selected from the group consisting of the AT₁ receptor antagonists, and the second active agent is selected from the group consisting of the diuretics.

7. The pharmaceutical preparation according to claim 1. wherein said pharmaceutical preparation comprises a combination of three active agents selected from the group consisting of the antihypertensive agents.

8. The pharmaceutical preparation according to claim 7, wherein one of the active agents is a beta receptor blocker, the second active agent is a vasodilator, and the third active agent is a diuretic, said vasodilator being selected from the group consisting of calcium antagonists, ACE inhibitors, alpha₁ receptor blockers and direct vasodilators.

9. The pharmaceutical preparation according to claim 7, characterised in that one of the active agents is an ACE inhibitor, the second active agent is a calcium antagonist, and the third active agent is a diuretic.

10. The pharmaceutical preparation according to claim 7, wherein one of the active agents is an antisympathotonic, the second active agent is a vasodilator, and the third active agent is a diuretic.

11. The pharmaceutical preparation according to claim 2, wherein the diuretic is selected from the group consisting of xanthine derivatives, osmotic diuretics, carbonic anhydrase inhibitors, thiazides, loop diuretics, potassium-sparing diuretics, aldosterone antagonists and cycloamidine derivatives.

12. The pharmaceutical preparation according to claim 2, wherein the active agents of the diuretics are selected from the group consisting of caffeine, theophylline, theobromine, mannite, sorbite, acetazolamide, hydrochlorothiazide, trichlormethiazide, butizide, bendroflumethiazide, bemetizide, mefruside, chlortalidone, xipamide, clopamide, indapamide, furosemide, azosemide, bumetanide, piretanide, torasemide, etozolin, etacrynic acid, methyl clothiazide, metolazone, polythiazide, spironolactone, potassium canrenoate, triamterene and amiloride, as well as pharmacologically acceptable salts and combinations of these active agents.

13. The pharmaceutical preparation according to claim 2, wherein the active agents of the beta receptor blockers are selected from the group consisting of alprenolol, oxprenolol, penbutolol, bupranolol, metipranolol, propanolol, nadolol, pindolol, mepindolol, carteolol, carazolol, timolol, sotalol, metoprolol, betaxolol, bisoprolol, atenolol, acebutolol, celiprolol and bopindolol, as well as pharmacologically acceptable salts and combinations of these active agents.

14. The pharmaceutical preparation according to claim 2, wherein the active agents of the alpha receptor blockers are selected from the group consisting of bunazosin, doxazosin, terazosin and urapidil, as well as pharmacologically acceptable salts and combinations of these active agents.

15. The pharmaceutical preparation according to claim 2, wherein the active agents of the ACE inhibitors are selected from the group consisting of benazepril, captopril, cilazapril, enalapril, fosinopril, imidapril, lisinopril, moexipril, perindopril, quinapril, ramipril, spirapril and trandolapril, as well as pharmacologically acceptable salts and combinations of these active agents.

16. The pharmaceutical preparation according to claim 2, wherein the calcium antagonists are selected from the group which consisting of calcium antagonists of the verapamil type, calcium antagonists of the diltiazem type and dihydropyridines.

17. The pharmaceutical preparation according to claim 2, wherein the active agents of the calcium antagonists are selected from the group consisting of diltiazem, gallopamil, verapamil, amlodipin, felodipine, isradipin, lacidipine, lercanidipin, nicardipine, nifedipine, nilvadipine, nisoldipine and nitrendipine, as well as pharmacologically acceptable salts and combinations of these active agents.

18. The pharmaceutical preparation according to claim 2, wherein the active agents of the AT₁ antagonists are selected from the group comprising consisting of candesartan, eprosartan, irbesartan, losartan, olmesartan, telmisartan and valsartan, as well as pharmacologically acceptable salts and combinations of these active agents.

19. The pharmaceutical preparation according to claim 10, wherein the active agents of the antisympathotonics are selected from the group consisting of clonidine and methyldopa as well as pharmacologically acceptable salts and combinations of these active agents.

20. The pharmaceutical preparation according to claim 2, wherein the active agents of the direct vasodilators are selected from the group consisting of minoxidil and dihydralazine as well as pharmacologically acceptable salts and combinations of these active agents.

21. The pharmaceutical preparation according to claim 1, wherein that the hydrophilic polymer is selected from the group consisting of dextran, polysaccharides, inclusive of starch and starch derivatives, cellulose derivatives, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of these polymers.

22. The pharmaceutical preparation according to claim 1, wherein the polymer film comprises a polyvinyl alcohol-polyethylene glycol graft copolymer.

23. The pharmaceutical preparation according to claim 1, further comprising a humectant selected from the group consisting of glycerine, propylene glycol, sorbitol, mannitol, polyethylene glycol and polyglycerol ester.

24. The pharmaceutical preparation according to claim 1, further comprising an antioxidant selected from the group consisting of vitamin C (ascorbic acid), ascorbyl palmitate, vitamin E (tocopherol acetate) and hydroxybenzoic acid derivatives.

25. The pharmaceutical preparation according to claim 1, wherein the active agent of the preparation is bound to an acidic or basic ion exchanger for taste masking.

26. The pharmaceutical preparation according to claim 1, further comprising dyes and/or pigments.

27. The pharmaceutical preparation according to claim 1 further comprising natural and/or synthetic flavouring substances.

28. The pharmaceutical preparation according to claim 1, further comprising the preparation contains a disintegrant or a wicking agent.

29. The pharmaceutical preparation according to claim 1, further comprising a buffer system for adjusting the pH value of the preparation.

30. The pharmaceutical preparation according to claim 1, wherein the hydrophilic polymer disintegrates within less than 5 minutes after application in the oral cavity of a user.

31. The pharmaceutical preparation according to claim 1, wherein the hydrophilic polymer disintegrates quickly in the oral cavity whereas the active agent remains bound to an ion exchanger which releases said active agent only upon reaching the gastrointestinal tract.

32. The pharmaceutical preparation according to claim 1, wherein the active agents are contained in discrete layers which are spatially separated from each other and which differ from each other in terms of the respective composition.

33. The pharmaceutical preparation according to claim 1, wherein the preparation is present as a foam having cavities and at least one of the active agents is present in liquid form within the cavities of said foam.

34. Use of a dosage form according to claim 1, for rectal, vaginal or intranasal administration of pharmaceutical active agents to humans or animals.

35. Use of an active agent combination according to claim 3 for the production of an oral dosage form which is based on hydrophilic polymers and which quickly disintegrates upon contact with moisture, for the treatment of high blood pressure, the dosage form comprising an active agent combination of at least two active agents which are suitable, for the treatment of hypertension.

36. The use according to claim 35, wherein the pharmaceutical product is formulated as a wafer.

37. A method for the therapeutic treatment of a person suffering from high blood pressure, comprising the step of orally administering a dosage form with transmucosal absorption, said dosage form being based on hydrophilic polymers and which quickly disintegrates upon contact with moisture, the dosage form comprising an active agent combination of antihypertensive agents, wherein one of the antihypertensive agents is selected from the group consisting of the beta receptor blockers, and the second antihypertensive agent is selected from the group consisting of the diuretics.

38. A method for the production of a sheet-like dosage form which is based on hydrophilic polymers and which quickly disintegrates upon contact with moisture, for the treatment of high blood pressure, the dosage form comprising an active agent combination of at least two active agents which are suitable for the treatment of hypertension, said method comprising the steps of:
preparing a solution which contains at least one polymer and at least two antihypertensive active agents;
spread-coating the solution on a coating substrate; and
solidifying the spread-coated solution by drying and withdrawing the solvent.

39. The pharmaceutical preparation according to claim 21, wherein said cellulose derivatives are selected from the group consisting of carboxymethyl cellulose, ethyl cellulose or propyl cellulose, hydroxypropylmethyl cellulose, hydroxypropyl cellulose, sodium carboxymethyl cellulose, methyl cellulose, hydroxyethyl cellulose and hydroxypropylethyl cellulose.

40. The pharmaceutical preparation according to claim 30, wherein the hydrophilic polymer disintegrates within less than 3 minutes after application in the oral cavity.

41. The pharmaceutical preparation according to claim 40, wherein the hydrophilic polymer disintegrates within less than 1 minute after application in the oral cavity.

42. The pharmaceutical preparation according to claim 41, wherein the hydrophilic polymer disintegrates within less than 30 seconds, after application in the oral cavity.
